(19) United States
(12) Patent Application Publication
(10) Pub. No.: US 2014/0336944 A1
(43) Pub. Date: Nov. 13, 2014

(54) INFORMATION PROCESSING APPARATUS, MOTION IDENTIFYING METHOD, AND RECORDING MEDIUM

(71) Applicants: Fumio YOSHIZAWA, Kanagawa (JP); Takeo TSUKAMOTO, Kanagawa (JP); Keisuke KONISHI, Kanagawa (JP)

(72) Inventors: Fumio YOSHIZAWA, Kanagawa (JP); Takeo TSUKAMOTO, Kanagawa (JP); Keisuke KONISHI, Kanagawa (JP)

(21) Appl. No.: 14/272,770

(22) Filed: May 8, 2014

(30) Foreign Application Priority Data
May 10, 2013 (JP) 2013-100283
Jan. 28, 2014 (JP) 2014-013503

(51) Int. Cl. A61B 5/11

(52) U.S. Cl. CPC A61B 5/1122; A61B 5/1116
USPC 702/19

(57) ABSTRACT

The present invention is concerning an information processing apparatus includes a record-information storage unit, a determining unit, a measuring unit, and an identifying unit. The record-information storage unit stores therein an already-identified person's motion together with time. The determining unit determines possible motions that a person can make from the already-identified person's motion. The measuring unit measures measurement information according to a person's motion. The identifying unit performs a pattern detecting process for detecting a pattern similar to the measurement information measured by the measuring unit in patterns corresponding to the possible motions determined by the determining unit out of predetermined patterns of measurement information for person's motions, and identifies a motion corresponding to the detected pattern as a motion that the person made.

### CROSS-REFERENCE TO RELATED APPLICATIONS

The present application claims priority to and incorporates by reference the entire contents of Japanese Patent Application No. 2013-100283 filed in Japan on May 10, 2013 and Japanese Patent Application No. 2014-013503 filed in Japan on Jan. 28, 2014.

### BACKGROUND OF THE INVENTION

1. Field of the Invention

The present invention relates to an information processing apparatus, a motion identifying method, and a computer-readable recording medium containing a motion identifying program.

2. Description of the Related Art

Conventionally, there is known a technology to identify a person's motion from temporal changes in the acceleration and angular velocity acting on the person's body. In the technology, patterns of measurement information, such as acceleration and angular velocity, and respective person's motions corresponding to the patterns are held in advance. Then, temporal changes in the acceleration and angular velocity acting on the body of a person who is subject to motion identification are compared with the previously-held patterns, and if a part similar to any of the patterns has been detected, a motion corresponding to the pattern is identified as a motion that the person made (see, for example, Japanese Patent No. 3570163, Japanese Patent Application Laid-open No. 2012-24449, and Japanese Patent Application Laid-open No. 2010-05033).

However, the above-described conventional technology has a problem that there is the potential for a decrease in processing performance. The conventional technology is useful in identifying one motion of a person with high accuracy; however, there are demands to identify more motions in practice. To identify more motions, comparisons with multiple patterns are performed; therefore, the processing load is increased, and time required to obtain a processing result is also increased. Consequently, the conventional technology holds the potential for a decrease in processing performance.

In view of the above, there is a need to provide an information processing apparatus, motion identifying method, and computer-readable recording medium containing a motion identifying program capable of suppressing a decrease in processing performance.

### SUMMARY OF THE INVENTION

It is an object of the present invention to at least partially solve the problems in the conventional technology.

According to the present invention, there is provided an information processing apparatus comprising: a determining unit configured to determine possible motions that a person can make; and an identifying unit configured to perform a pattern detecting process for detecting a pattern similar to measurement information measured according to a person's motion in patterns corresponding to the possible motions determined by the determining unit out of predetermined patterns of measurement information for person's motions, and identify a motion corresponding to the detected pattern as a motion that the person made.

The present invention also provides a motion identifying method comprising: determining possible motions that a person can make; and performing a pattern detecting process for detecting a pattern similar to measurement information measured according to a person's motion in patterns corresponding to the possible motions determined at the determining out of predetermined patterns of measurement information for person's motions, and identifying a motion corresponding to the detected pattern as a motion that the person made.

The present invention also provides a non-transitory computer-readable recording medium that contains a motion identifying program causing a computer to execute: determining possible motions that a person can make; and performing a pattern detecting process for detecting a pattern similar to measurement information measured according to a person's motion in patterns corresponding to the possible motions determined at the determining out of predetermined patterns of measurement information for person's motions, and identifying a motion corresponding to the detected pattern as a motion that the person made.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating an application example of an information processing apparatus;

FIG. 2 is a functional block diagram showing a configuration example of an information processing apparatus according to a first embodiment of the present invention;

FIG. 3 is a diagram showing an example of record information according to the first embodiment;

FIG. 4 is a diagram showing an example of correspondence information according to the first embodiment;

FIG. 5 is a diagram illustrating a coordinate system representing the respective magnitude and directions of acceleration, angular velocity, and geomagnetic field;

FIG. 6 is a diagram showing an example of respective waveforms of acceleration and angular velocity measured by a measuring unit;

FIG. 7 is a diagram showing an example of pattern information on output waveform patterns of acceleration and angular velocity according to person's motion;

FIG. 8 is a flowchart showing an example of the flow of a motion identifying process according to the first embodiment;

FIG. 9 is a functional block diagram showing a configuration example of an information processing apparatus according to a second embodiment;

FIG. 10 is a diagram showing an example of map information according to the second embodiment;

FIG. 11 is a diagram showing an example of correspondence information according to the second embodiment;

FIG. 12 is a diagram showing an example of a correlation chart for creating the correspondence information according to the second embodiment;

FIG. 13 is a diagram showing an example of a predetermined range of area including person's present location according to the second embodiment;

FIG. 14 is a flowchart showing an example of the flow of a motion identifying process according to the second embodiment; and

FIG. 15 is a diagram illustrating an example of directions of acceleration and angular velocity.

### DETAILED DESCRIPTION OF THE PREFERRED EMBODIMENTS

Exemplary embodiments of an information processing apparatus, motion identifying method, and motion identifying program according to the present invention will be explained below with reference to accompanying drawings. Incidentally, the present invention is not limited to the embodiments described below. Furthermore, the embodiments can be arbitrarily combined within a scope which does not contradict contents.

### First Embodiment

Application Example of Information Processing Apparatus

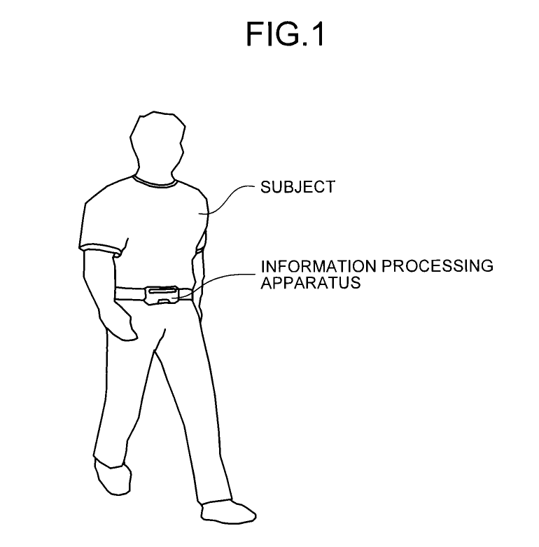

An application example of an information processing apparatus according to a first embodiment is explained with FIG. 1. FIG. 1 is a diagram illustrating the application example of the information processing apparatus.

As shown in FIG. 1, the information processing apparatus is information equipment fitted on a subject (a person) who is subject to motion identification. The body part fitted with the information processing apparatus is, for example, the abdomen which is the center of gravity of the human body. When the information processing apparatus is fitted on the abdomen, the acceleration and angular velocity acting on the gravity center of the human body can be measured. Incidentally, the fitting of the information processing apparatus on the abdomen is just an example, and the body part fitted with the information processing apparatus varies according to content of body information that one wants to measure.

Configuration of Apparatus According to First Embodiment

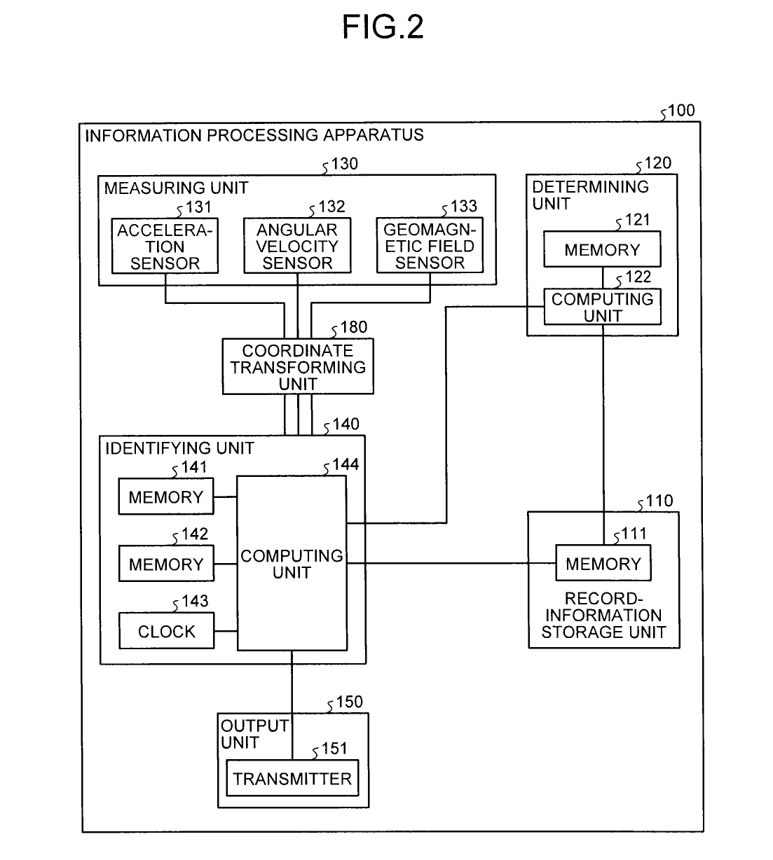

Subsequently, a configuration of an information processing apparatus according to the first embodiment is explained with FIG. 2. FIG. 2 is a functional block diagram showing a configuration example of the information processing apparatus according to the first embodiment.

As shown in FIG. 2, an information processing apparatus 100 includes a record-information storage unit 110, a determining unit 120, a measuring unit 130, an identifying unit 140, an output unit 150, and a coordinate transforming unit 180.

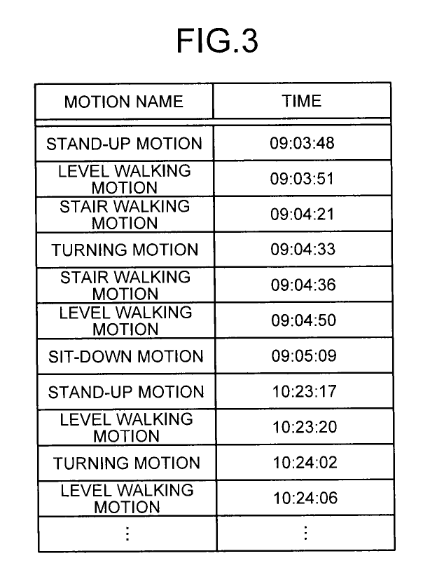

The record-information storage unit 110 stores therein record information on a recorded person's motion. The record-information storage unit 110 includes a memory 111. Specifically, the memory 111 stores therein, as record information, a motion name of a person's motion identified by the identifying unit 140 and the time of the identification. FIG. 3 is a diagram showing an example of record information according to the first embodiment. As shown in FIG. 3, the record information is information that associates motion name with time. To take some record information as an example, record information that associates motion name “stand-up motion” with time “09:03:48” and record information that associates motion name “level walking motion” with time “09:03:51” exist. From the example shown in FIG. 3, we can see a person's motion history that a person stood up at 09:03:48 and then started level walking at 09:03:51.

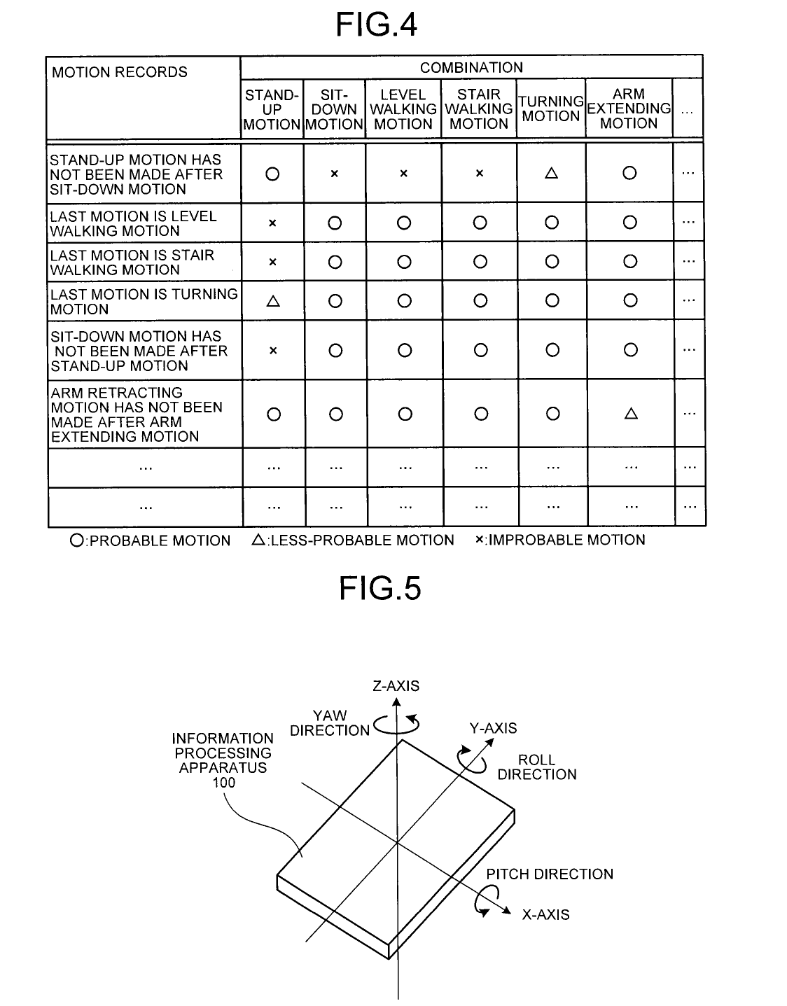

The determining unit 120 determines a person's possible motion. The determining unit 120 includes a memory 121 and a computing unit 122. The memory 121 stores therein correspondence information indicating correspondence of a person's motion to person's next possible motions that the person can make after the motion. Specifically, the memory 121 stores therein correspondence information on correspondence of a person's motion to the next possible motions based on a person's state or a sequence of person's motions, etc. FIG. 4 is a diagram showing an example of correspondence information according to the first embodiment. As shown in FIG. 4, the correspondence information is information that classifies motions combined with a motion record according to probability. In the example shown in FIG. 4, a probable motion is denoted by a circle mark, a less-probable motion is denoted by a triangle mark, and an improbable motion is denoted by a cross mark.

To take correspondence information of a motion record “a stand-up motion has not been made after a sit-down motion” as an example, the motion record is associated with stand-up motion “a circle mark”, sit-down motion “a cross mark”, level walking motion “a cross mark”, stair walking motion “a cross mark”, turning motion “a triangle mark”, and arm extending motion “a circle mark”, etc. That is, when a person is in a seated state, a stand-up motion or an arm extensional motion is probably made, a turning motion is less probably made, and the other motions are improbably made.

Such correspondence information is created on the basis of a person's state or a sequence of person's motions as described above. First, a case where correspondence information is created on the basis of a person's state is explained. For example, when a person is in a seated state in a chair, the person is unlikely to make a motion of walking around or a motion of going up and down stairs. Therefore, during a period of time from when the person has made a sit-down motion until the person makes a stand-up motion next, a level walking motion and a stair walking motion are “improbable motions”. Furthermore, for example, when a person is in a seated state in a chair, the person rarely turns. Therefore, during a period of time from when the person has made a sit-down motion until the person makes a stand-up motion next, a turning motion is a “less-probable motion”. Moreover, for example, when a person is in a standing state, the person is unlikely to further stand up. Therefore, during a period of time from when the person has made a motion which can be interpreted as the person standing (for example, a level walking motion or a stair walking motion, etc.) until the person makes a sit-down motion next, a stand-up motion is an “improbable motion”. In short, it is only necessary to think whether each of the person's next possible motions is a contradictory motion or not on the basis of the current person's state.

Next, a case where correspondence information is created on the basis of a sequence of person's motions is explained. For example, consecutive stand-up motions do not occur. Also, consecutive sit-down motions do not occur. In other words, a stand-up motion and a sit-down motion are motions that alternately occur, and neither of the motions occurs consecutively. Therefore, during a period of time from when a person has made a stand-up motion until the person makes a sit-down motion, a stand-up motion is an “improbable motion”. Also, during a period of time from when a person has made a sit-down motion until the person makes a stand-up motion, a sit-down motion is an “improbable motion”. Furthermore, for example, the arm length is finite, so it is rare that only an arm extending motion is consecutively made several times. Therefore, during a period of time from when a person has made an arm extending motion until the person makes an arm retracting motion next, an arm extending motion is a “less-probable motion”. In short, it is only necessary to think whether each of the person's next possible motions is a contradictory motion as a sequence of person's motions. Incidentally, correspondence information is created as described above; however, one kind of correspondence information is not always applicable to everyone, so it is preferable to use different correspondence information for each subject.

To return to the explanation of FIG. 2, the computing unit 122 determines person's possible motions from an already-identified person's motion on the basis of the correspondence information. Specifically, the computing unit 122 sequentially refers to record information stored in the memory 111 and determines person's next possible motions in accordance with the correspondence information stored in the memory 121. The possible motions here correspond to “probable motion” and “less-probable motion” shown in FIG. 4. To explain the possible motions with the example shown in FIG. 4, when a person is in a seated state based on record information, a “stand-up motion: a circle mark”, a “turning motion: a triangle mark”, and an “arm extending motion: a circle mark” are the next possible motions. Then, the computing unit 122 outputs the determined possible motions to the identifying unit 140.

The measuring unit 130 measures measurement information. The measuring unit 130 includes an acceleration sensor 131, an angular velocity sensor 132, and a geomagnetic field sensor 133. The acceleration sensor 131 measures the magnitude and direction of acceleration acting on the information processing apparatus 100 as a piece of measurement information. Specifically, the acceleration sensor 131 measures the magnitude and direction of acceleration acting on the information processing apparatus 100 at regular intervals, and outputs X, Y, and Z components of the measured acceleration as digital values to the coordinate transforming unit 180. The angular velocity sensor 132 measures the magnitude and direction of rotational speed of the information processing apparatus 100 as a piece of measurement information. Specifically, the angular velocity sensor 132 measures the magnitude and direction of rotational speed of the information processing apparatus 100 at regular intervals, and outputs pitch, roll, and yaw components of the measured rotational speed as digital values to the coordinate transforming unit 180. The geomagnetic field sensor 133 measures the magnitude and direction of geomagnetic field near the information processing apparatus 100 as a piece of measurement information. Specifically, the geomagnetic field sensor 133 measures the magnitude and direction of geomagnetic field near the information processing apparatus 100 at regular intervals, and outputs X, Y, and Z components of the measured geomagnetic field as digital values to the coordinate transforming unit 180.

FIG. 5 is a diagram illustrating a coordinate system representing the respective magnitude and directions of acceleration, angular velocity, and geomagnetic field. As shown in FIG. 5, respective X, Y, and Z components of the acceleration and the geomagnetic field correspond to X-axis, Y-axis, and Z-axis directions, respectively. Furthermore, the pitch direction of the angular velocity corresponds to a direction of rotating about the X-axis, the roll direction corresponds to a direction of rotating about the Y-axis, and the yaw direction corresponds to a direction of rotating about the Z-axis.

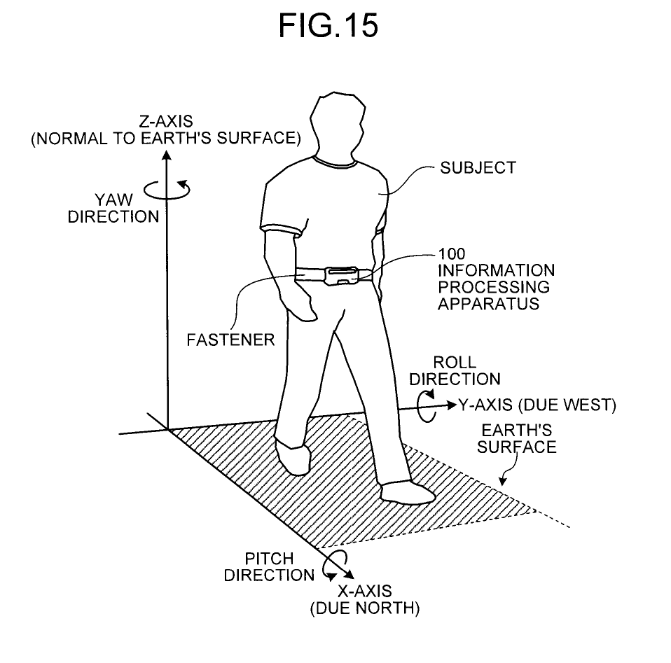

The coordinate transforming unit 180 finds out which direction of the information processing apparatus 100 is the direction of gravity and further finds out which direction of the information processing apparatus 100 is the direction of magnetic north on the basis of measurement information, and performs coordinate transformation of the measurement information. Specifically, the coordinate transforming unit 180 finds out the direction of gravity from the direction of gravitational acceleration acting on the information processing apparatus 100, and finds out the direction of magnetic north from the direction of geomagnetic field acting on the information processing apparatus 100. Then, the coordinate transforming unit 180 transforms the found directions of gravity and magnetic north direction into components corresponding to X-axis, Y-axis, and Z-axis directions of a coordinate system based on the earth's surface as shown in FIG. 15, and outputs a result of the transformation to the identifying unit 140.

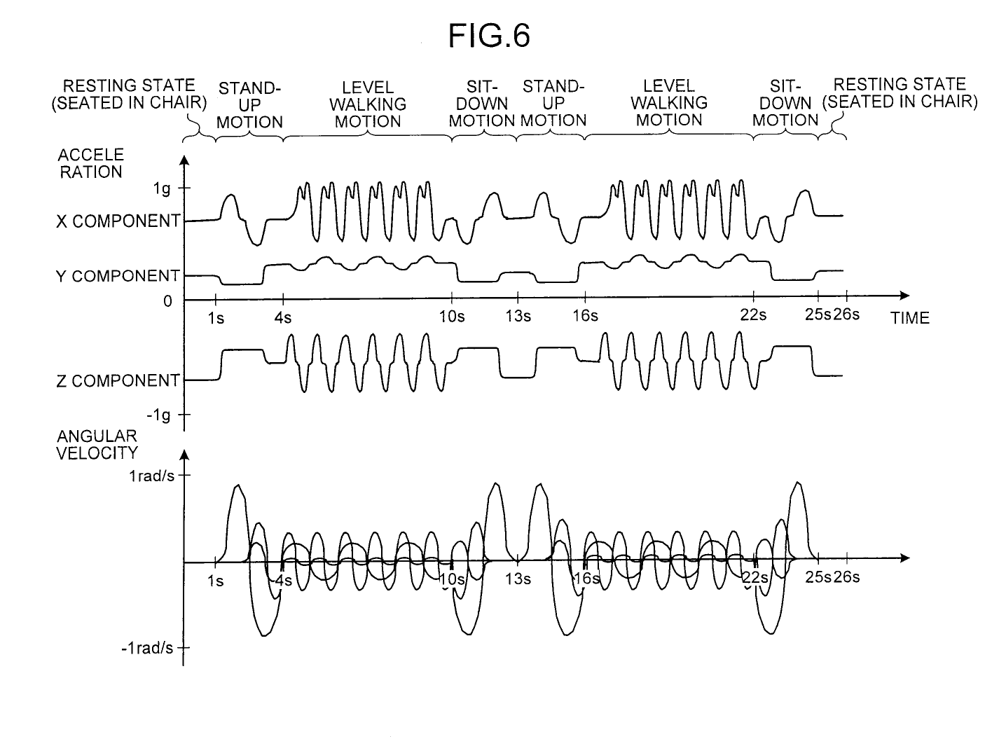

FIG. 6 is a diagram showing an example of respective waveforms of the acceleration and angular velocity measured by the measuring unit 130. In the example shown in FIG. 6, there is shown waveforms output when a person in a chair made motions of “standing up from the chair and walking on the flat floor, and then again sitting down in the chair” twice repeatedly. As shown in FIG. 6, while the person is seated in the chair (from 0 s to 1 s and from 25 s to 26 s), the acceleration sensor 131 outputs a fixed value, and the angular velocity sensor 132 outputs 0. That is, while the person is seated in the chair, the center of gravity of the person does not move; therefore, the acceleration sensor 131 outputs a fixed value, and the angular velocity sensor 132 outputs 0. Only X, Y, and Z components of gravitational acceleration are output from the acceleration sensor 131.

Furthermore, when the person made the stand-up motions (from 1 s to 4 s and from 13 s to 16 s), similar output waveforms appear in the both time periods. Also, when the person made the walking motions (from 4 s to 10 s and from 16 s to 22 s), similar output waveforms appear in the both time periods. Also, when the person made the sit-down motions (from 10 s to 13 s and from 22 s to 25 s), similar output waveforms appear in the both time periods. In short, when a person makes the same motion, similar output waveforms appear because there is regularity in the movement of the center of gravity. Furthermore, the regularity in the movement of the center of gravity differs according to person's motion. Accordingly, if the regularity in the movement of the center of gravity is found, a person's motion can be identified from respective output waveforms output from the acceleration sensor 131 and the angular velocity sensor 132.

To return to the explanation of FIG. 2, the identifying unit 140 identifies a person's motion. The identifying unit 140 includes a memory 141, a memory 142, a clock 143, and a computing unit 144. The memory 141 temporarily stores therein a measured value (a digital value) of acceleration measured by the acceleration sensor 131 and a measured value (a digital value) of angular velocity measured by the angular velocity sensor 132. The memory 142 stores therein pattern information on output waveform patterns of acceleration and angular velocity according to person's motion. As an example, the memory 142 stores therein an average value, the maximum value, the minimum value, and a differential value, etc. of an output waveform. The clock 143 outputs current time to the computing unit 144.

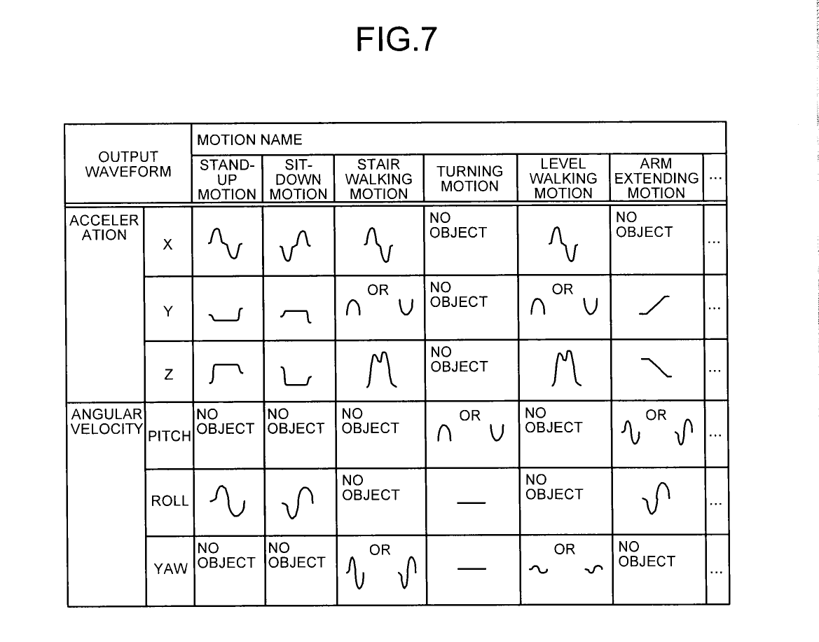

FIG. 7 is a diagram showing an example of pattern information on output waveform patterns of acceleration and angular velocity according to person's motion. As shown in FIG. 7, the pattern information is information that associates a motion name of a person's motion with output waveforms of the acceleration and angular velocity corresponding to the motion name. If obtained output waveforms are similar to any combination of output waveforms of components of acceleration and angular velocity shown in FIG. 7, it shall be considered that a person made a corresponding motion. As an example, if an average value, the maximum value, the minimum value, and a differential value, etc. of an output waveform are similar to any of those shown in FIG. 7, it can be considered that a person made a corresponding motion.

The computing unit 144 identifies a person's motion. Specifically, the computing unit 144 receives the next possible motions determined by the computing unit 122. Furthermore, the computing unit 144 receives digital values of acceleration measured by the acceleration sensor 131 and digital values of angular velocity measured by the angular velocity sensor 132. Then, the computing unit 144 temporarily stores the digital values of acceleration and the digital values of angular velocity in the memory 141, and reproduces respective output waveforms of the acceleration and angular velocity.

Then, the computing unit 144 attempts detection of a similar pattern by comparing temporal changes in the reproduced output waveforms with respective pieces of pattern information in the memory 142 that correspond to the next possible motions. Specifically, when possible motions determined by the computing unit 122 are a “stand-up motion”, a “turning motion”, and an “arm extending motion”, the computing unit 144 detects a pattern similar to temporal changes in output waveforms by referring to only respective pieces of pattern information corresponding to these possible motions. In this case, as for a “sit-down motion”, a “level walking motion”, and a “stair walking motion”, a pattern detecting process for identifying a motion is not performed. If the computing unit 144 has detected a similar pattern, the computing unit 144 identifies a motion corresponding to the similar pattern as a motion that a person made. After that, the computing unit 144 outputs a motion name of the person's motion and current time obtained from the clock 143 to the output unit 150. Furthermore, the computing unit 144 stores the motion name of the person's motion and the current time in the memory 111.

The output unit 150 outputs a processing result of a process performed by the information processing apparatus 100. The output unit 150 includes a transmitter 151. The transmitter 151 transmits a motion name of a person's motion and current time. Specifically, the transmitter 151 transmits the person's motion name and current time output from the computing unit 144 to an external device by wireless communication, etc. As a wireless communication system, for example, Bluetooth™ or Wi-Fi™ (Wireless Fidelity), etc. is adopted.

Flow of Motion Identifying Process According to First Embodiment

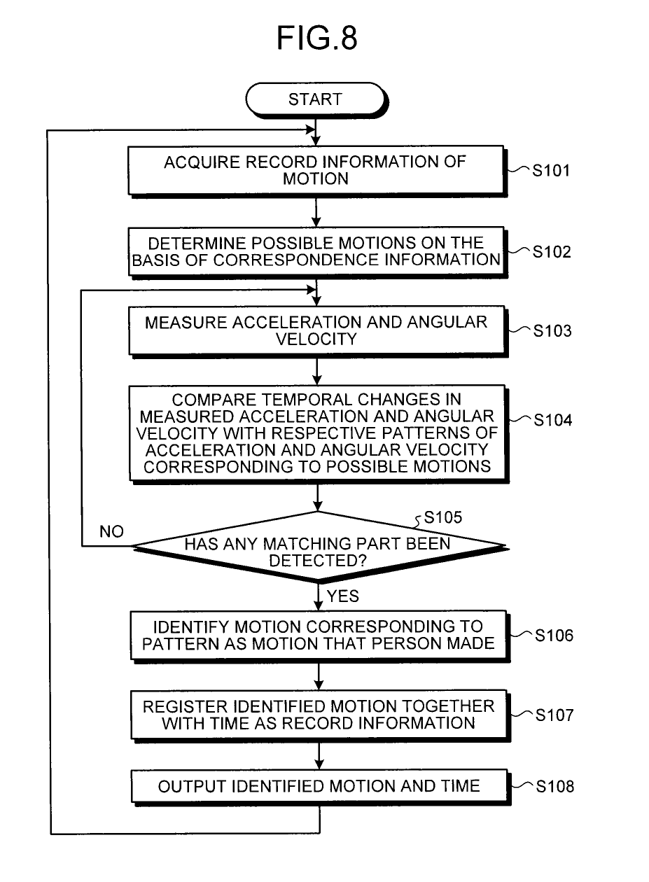

Subsequently, the flow of a motion identifying process according to the first embodiment is explained with FIG. 8. FIG. 8 is a flowchart showing an example of the flow of the motion identifying process according to the first embodiment.

As shown in FIG. 8, the computing unit 122 acquires record information of an already-identified person's motion stored in the memory 111 (Step S101). Then, the computing unit 122 determines person's next possible motions from the acquired record information in accordance with the correspondence information stored in the memory 121 (Step S102). The acceleration sensor 131 and the angular velocity sensor 132 measure acceleration and angular velocity, respectively (Step S103).

The computing unit 144 compares temporal changes in the acceleration and angular velocity measured by the acceleration sensor 131 and the angular velocity sensor 132 with respective output waveform patterns of acceleration and angular velocity corresponding to the possible motions determined by the computing unit 122 with reference to the memory 142 (Step S104). As an example, the computing unit 144 compares an average value, the maximum value, the minimum value, and a differential value, etc. of an output waveform. When the computing unit 144 has detected a part similar to any of the patterns (YES at Step S105), the computing unit 144 identifies a motion corresponding to the similar pattern as a motion that the person made (Step S106). On the other hand, if the computing unit 144 has not detected any part similar to any of the patterns (NO at Step S105), that means the record information of person's motion remains unchanged, so the process at Step S103 is again performed.

Then, the computing unit 144 registers, as record information, a motion name of the identified motion together with current time obtained from the clock 143 on the memory 111 (Step S107). The transmitter 151 transmits the motion name of the motion identified by the computing unit 144 and the current time to an external device (Step S108). Incidentally, such a motion identifying process is repeatedly performed.

Effect of First Embodiment

The information processing apparatus 100 determines person's next possible motions from an already-identified person's motion, and compares temporal changes in measured measurement information with respective patterns of measurement information corresponding to the next possible motions, and, when having detected a part similar to any of the patterns, identifies a motion corresponding to the pattern as a motion that the person made. The information processing apparatus 100 targets only patterns of measurement information corresponding to the next possible motions for comparison with temporal changes in measured measurement information, and consequently can suppress a decrease in processing performance. In other words, even when the number of patterns to be compared with temporal changes in measurement information (the number of motions to be identified) is increased, the information processing apparatus 100 can suppress a decrease in processing performance as compared with the conventional technology that targets all patterns for comparison. If there are ten motions to be identified, and it takes 1 microsecond to identify each motion, it takes 10 microseconds to compare all patterns with temporal changes in measurement information; however, if the next possible motions are three motions, it takes only 3 microseconds.

Variation of First Embodiment

In the first embodiment described above, the motion identifying process that targets patterns corresponding to the next possible motions for comparison with temporal changes in measurement information is explained. In a variation of the first embodiment, there is explained a case where the motion identifying process is performed according to probability of a possible motion.

The variation of the first embodiment is explained with FIG. 4. As explained in the first embodiment, in the example shown in FIG. 4, when a person is in a seated state, a “stand-up motion: a circle mark”, a “turning motion: a triangle mark”, and an “arm extending motion: a circle mark” are the next possible motions. Out of the next possible motions, a “stand-up motion” and an “arm extending motion” are probable motions, and a “turning motion” is a less-probable motion. That is, even though these motions are the next possible motions, the motions differ in probability. From this aspect, in the variation of the first embodiment, the motion identifying process is performed according to probability of the next possible motion.

Specifically, when the computing unit 122 outputs the next possible motions to the identifying unit 140, the computing unit 122 further outputs respective incidence rates that represent the degrees of probability of the possible motions. Being a “probable motion” or being a “less-probable motion” is an example of an incidence rate of a possible motion. A “probable motion” has a higher incidence rate than a “less-probable motion”. That is, the computing unit 122 outputs information that when a person is in a seated state, “a stand-up is a probable motion”, “a turning motion is a less-probable motion”, and “an arm extending motion is a probable motion” to the identifying unit 140.

Furthermore, in the identifying unit 140, the computing unit 144 performs the pattern detecting process so that the lower the incidence rate of a possible motion output from the computing unit 122 is, the more simplified pattern detecting process the computing unit 144 performs. As a method for achieving the simplification of the pattern detecting process, for example, a method of replacing a program for the pattern detecting process or a method of replacing setup information called parameters of the program can be used. Furthermore, as for a possible motion having a low incidence rate, the process can be omitted instead of performing the pattern detecting process in a simplified manner. To explain with the above-described example, the computing unit 144 performs the process using pattern information corresponding to a “stand-up motion” or “arm extending motion” which is a probable motion, and performs the process on a “turning motion” which is a less-probable motion in a more simplified manner than a probable motion. The other functions other than these are the same as the first embodiment, so description of the other functions is omitted.

Effect of Variation of First Embodiment

Depending on the probability of a person's next possible motion, the information processing apparatus 100 simplifies the pattern detecting process corresponding to a less-probable motion, and therefore can suppress a decrease in processing performance as compared with a case where only the pattern detecting process corresponding to an improbable motion is omitted. If there are ten motions to be identified, and it takes 2 microseconds to perform one conventional pattern detecting process and 1 microsecond to perform one simplified pattern detecting process, it takes 20 microseconds to perform the conventional pattern detecting process on all patterns; however, if out of the ten motions, five are less-probable motions, five pattern detecting processes can be simplified, so it takes only 15 microseconds.

### Second Embodiment

In the first embodiment, there is described the case where the next possible motions are determined on the basis of correspondence information created based on a person's state or a sequence of person's motions. In a second embodiment, there is described a case where the next possible motions are determined on the basis of correspondence information indicating correspondence of a thing or another person located around a person to a motion that the person makes to the thing or another person. Incidentally, an application example of an information processing apparatus according to the second embodiment is the same as the first embodiment.

Configuration of Apparatus According to Second Embodiment

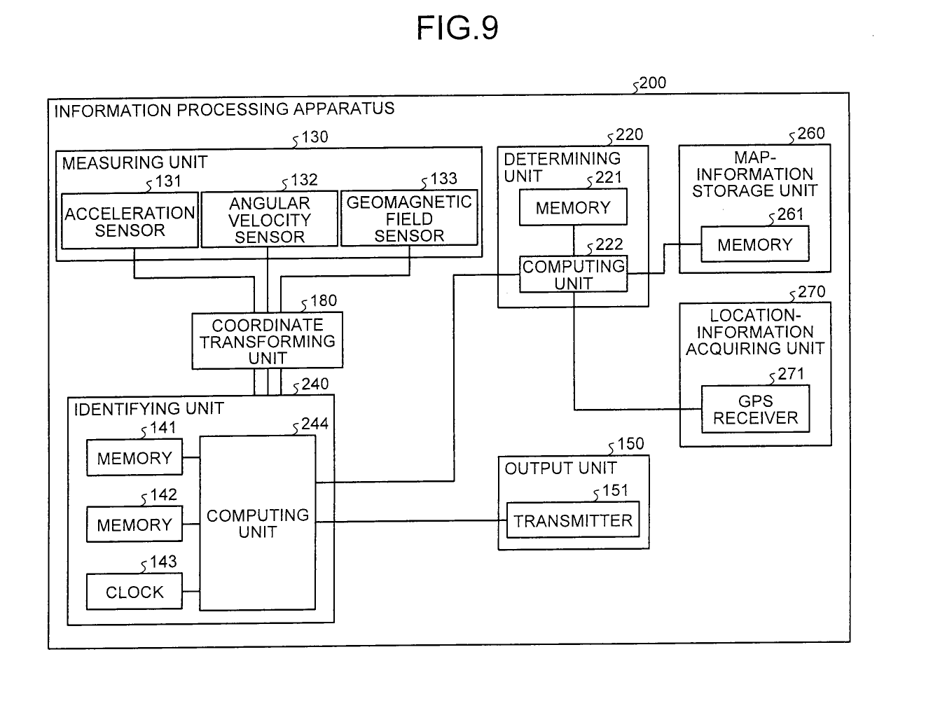

A configuration of the information processing apparatus according to the second embodiment is explained with FIG. 9. FIG. 9 is a functional block diagram showing a configuration example of the information processing apparatus according to the second embodiment. In the second embodiment, a component identical to that in the first embodiment is assigned the same reference numeral, and detailed description of the component may be omitted. Specifically, the functions and configurations of the measuring unit 130, the output unit 150, and the coordinate transforming unit 180 mentioned below and processes performed by them are the same as those described in the first embodiment.

As shown in FIG. 9, an information processing apparatus 200 includes a determining unit 220, the measuring unit 130, an identifying unit 240, the output unit 150, the coordinate transforming unit 180, a map-information storage unit 260, and a location-information acquiring unit 270.

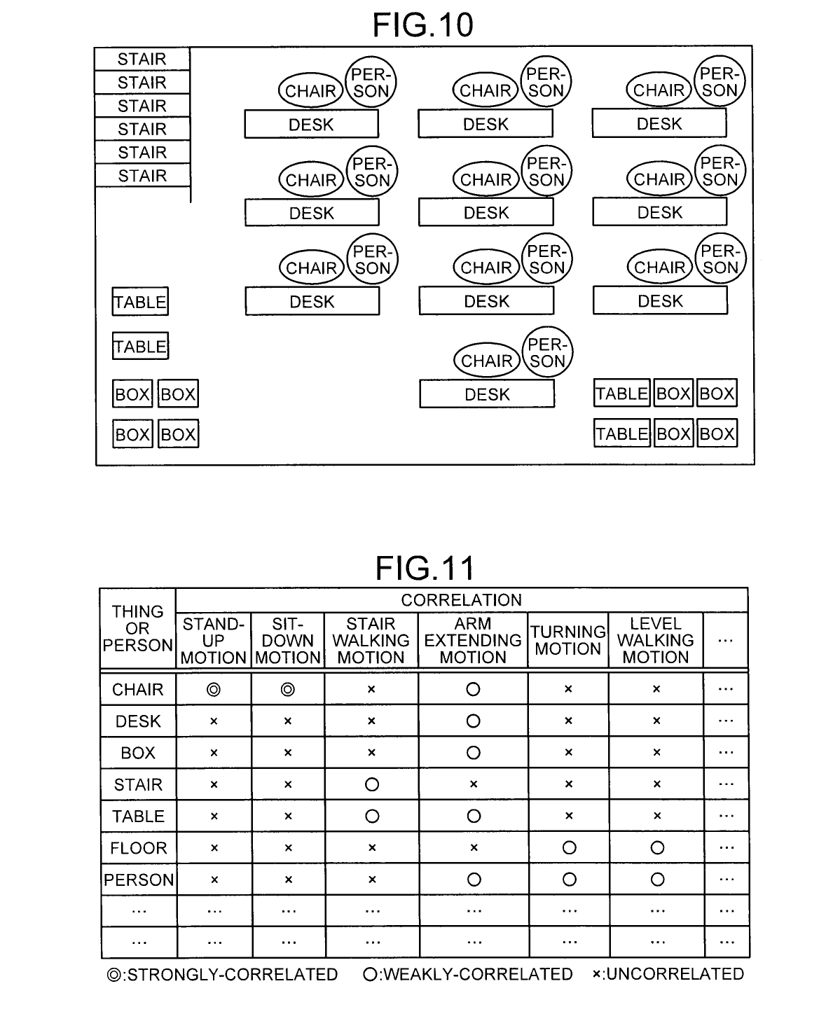

The map-information storage unit 260 stores therein map information. The map-information storage unit 260 includes a memory 261. Specifically, the memory 261 stores therein map information of an activity area of a person who is subject to motion identification. The map information represents not only a map but also things and/or other persons located therein. For example, if a person subject to motion identification is a hospitalized patient, the floors of the hospital is a person's activity area, so a floor map of the hospital is used as map information. Furthermore, for example, if a person subject to motion identification is a corporate employee, the floor of person's office is a person's activity area, so a floor map of the office is used as map information. FIG. 10 is a diagram showing an example of map information according to the second embodiment. As shown in FIG. 10, the map information is a map of a floor in an activity area of a person who is subject to motion identification and information of things and/or other persons located on the floor. The things include, for example, stairs, tables, boxes, desks and chairs, etc. located on the floor. The other persons are, for example, persons seated in chairs, etc.

The location-information acquiring unit 270 acquires location information. The location-information acquiring unit 270 includes a global positioning system (GPS) receiver 271. Specifically, the GPS receiver 271 receives a GPS signal from a GPS satellite, and outputs the received GPS signal as location information. The location information represents the present location of a person subject to motion identification. As a system of GPS, for example, publicly-known technologies, such as IMES (Indoor Messaging System) and NFC (Near Field Communication), can be used.

The determining unit 220 determines a person's possible motion. The determining unit 220 includes a memory 221 and a computing unit 222. The memory 221 stores therein correspondence information indicating correspondence of a thing or another person to a motion that a person makes to the thing or another person. Specifically, the memory 221 stores therein correlation between a thing or another person and the next possible motions based on possible motions that a person may make to the thing or another person. FIG. 11 is a diagram showing an example of correspondence information according to the second embodiment. As shown in FIG. 11, the correspondence information is information that classifies combinations of a thing or another person and motions made to the thing or another person according to probability. In FIG. 11, the probability is expressed in correlation. In the example shown in FIG. 11, a strongly-correlated motion is denoted by “a double circle mark”, a weakly-correlated motion is denoted by “a circle mark”, and an uncorrelated motion is denoted by “a cross mark”.

To take correspondence information of a thing “chair” as an example, the thing “chair” is associated with stand-up motion “a double circle mark”, sit-down motion “a double circle mark”, stair walking motion “a cross mark”, arm extending motion “a circle mark”, turning motion “a cross mark”, and level walking motion “a cross mark”, etc. That is, as a motion that a person makes to a chair, a stand-up motion and a sit-down motion are probable because these motions correlate strongly with a chair, an arm extending motion is less probable because this motion correlates weakly with a chair, and the other motions are improbable because the other motions are uncorrelated with a chair. In the correspondence information according to the first embodiment (see FIG. 4), an “arm extending motion” is a possible motion when a person is in a seated state. However, in the correspondence information according to the second embodiment, the “arm extending motion” is not a motion of extending person's arm in a state where a person is being seated but a motion of putting person's hand on a thing “chair” to lift and carry the chair; therefore, the correspondence information according to the second embodiment differs in intent of motion from that of the first embodiment.

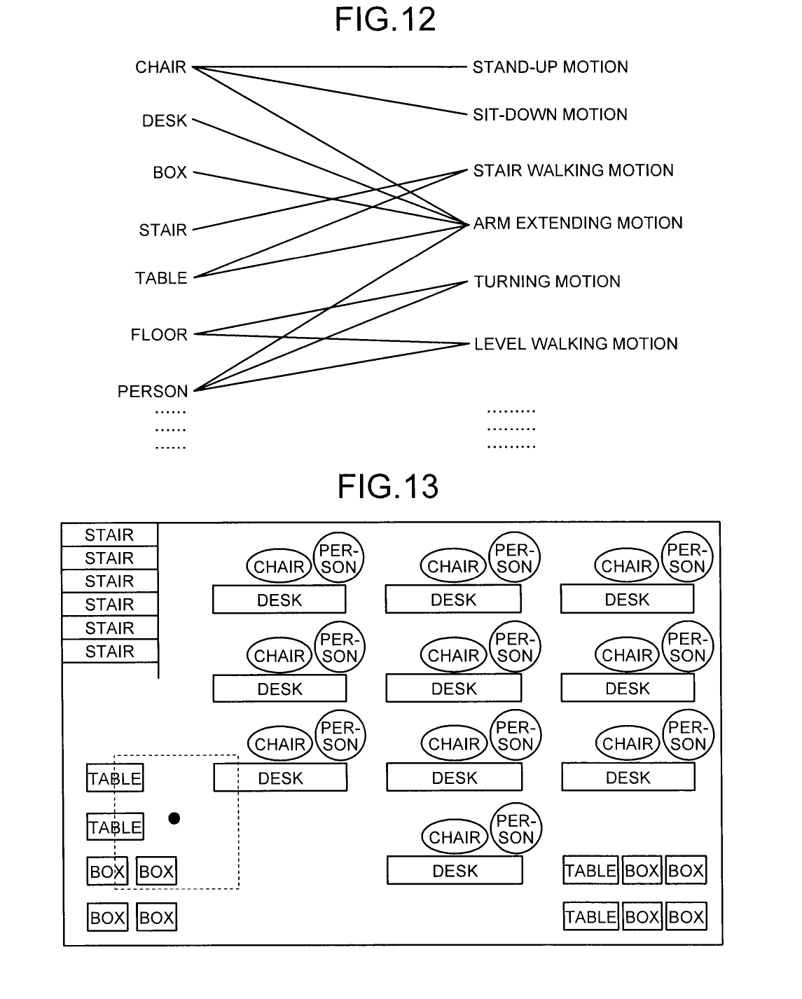

Such correspondence information is created on the basis of a correlation chart. FIG. 12 is a diagram showing an example of a correlation chart for creating the correspondence information according to the second embodiment. First, write down the things and/or other persons included in the map information (see FIG. 10) and motion names of motions that a person makes as shown in FIG. 12. Then, connect each of the things and/or other persons and motion name(s) of correlated motion(s) with line(s). For example, motion names of motions correlated with a chair include a stand-up motion, a sit-down motion, and an arm extending motion, etc.; therefore, the chair and these motions are connected with lines. Then, connect each of motion names and things and/or other persons that can be objects of a motion corresponding to the motion name with lines. For example, objects of a stair walking motion include things that make a difference in level, such as stairs and a table; therefore, the stair walking motion and these things are connected with lines. Furthermore, for example, objects of an arm extending motion include things that can be carried by hand(s), such as a chair, a desk, a box, and a table; therefore, the arm extending motion and these things are connected with lines. Moreover, if another person is around, a person can do an action, such as approach another person, move away from another person, or hand a thing to another person; therefore, (another) person and a level walking motion, a turning motion, and an arm extending motion are connected with lines. Accordingly, the correlation chart shown in FIG. 12 is created.

After that, as for a motion name connected to only one thing or person, both sides shall be deemed to have a strong correlation. For example, “stand-up motion” and “sit-down motion” connected to only “chair” have a strong correlation with a chair. Furthermore, as for a motion name connected to multiple things and/or other persons, both sides shall be deemed to have a weak correlation. For example, “stair walking motion” is connected to multiple things such as “stair” and “table”, and therefore shall be deemed to have a weak correlation with “stair” and “table”. Moreover, a thing and a motion name, which are not connected to each other, shall be deemed to be uncorrelated. For example, “table” and “sit-down motion” are not connected to each other, and therefore shall be deemed to be uncorrelated.

To return to the explanation of FIG. 9, the computing unit 222 determines the next possible motions that a person can make to a thing or another person located in a predetermined range of area including person's present location in map information. Specifically, the computing unit 222 refers to the map information stored in the memory 261 and detects thing(s) and/or other person(s) located in the predetermined range of area including the person's present location output from the GPS receiver 271. The predetermined range of area including the person's present location shall be a range of area that a person can reach by stretching out his/her arm or leg in one motion. FIG. 13 is a diagram showing an example of the predetermined range of area including the person's present location according to the second embodiment. In the example shown in FIG. 13, the person's present location is indicated by a black circle. For example, as shown in FIG. 13, the predetermined range of area including the person's present location is a square area with two meters on each side centering around the person's present location (the black circle). In the example shown in FIG. 13, the computing unit 222 detects things, such as “tables”, “boxes”, and a “desk”, and/or other persons located in the predetermined range of area including the person's present location.

Then, the computing unit 222 determines the next possible motions that the person can make to the detected things and/or other persons on the basis of the correspondence information stored in the memory 221. The possible motions here correspond to “strongly-correlated” motions and “weakly-correlated” motions shown in FIG. 11. To explain the possible motions with the example shown in FIG. 11, when a “chair” is included in the predetermined range of area including the person's present location, a “stand-up motion: a double circle mark”, a “sit-down motion: a double circle mark”, and an “arm extending motion: a circle mark” are the next possible motions. Then, the computing unit 222 outputs the determined possible motions to the identifying unit 240. Incidentally, just like in the variation of the first embodiment, the computing unit 222 can further output respective probabilities (incidence rates) of the possible motions.

The identifying unit 240 identifies a person's motion. The identifying unit 240 includes the memory 141, the memory 142, the clock 143, and a computing unit 244. The memory 141, the memory 142, and the clock 143 are the same as those in the first embodiment. The computing unit 244 differs from the computing unit 144 according to the first embodiment in that the computing unit 244 does not store an identified person's motion as record information in the memory. That is, the computing unit 244 receives possible motions determined by the computing unit 222 and measurement information measured by the measuring unit 130, and detects a similar pattern by referring to pattern information stored in the memory 142, thereby identifying a person's motion. Incidentally, just like in the variation of the first embodiment, the computing unit 244 can perform a motion identifying process according to probability of a possible motion.

Flow of Motion Identifying Process According to Second Embodiment

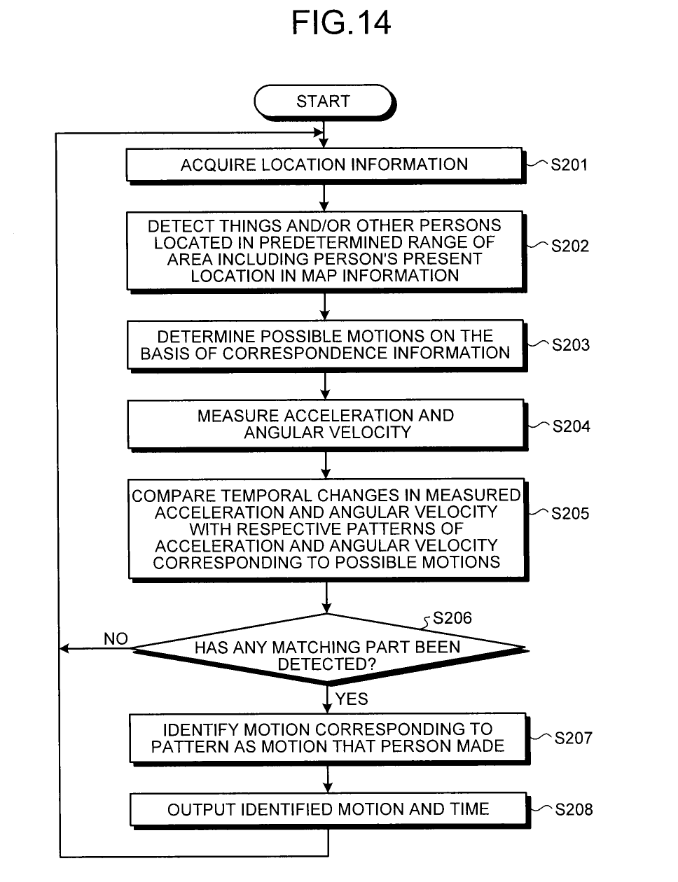

Subsequently, the flow of the motion identifying process according to the second embodiment is explained with FIG. 14. FIG. 14 is a flowchart showing an example of the flow of the motion identifying process according to the second embodiment.

As shown in FIG. 14, the computing unit 222 acquires location information from the GPS receiver 271 (Step S201). Then, the computing unit 222 detects thing(s) and/or other person(s) located in a predetermined range of area including person's present location based on the acquired location information by referring to map information stored in the memory 261 (Step S202). Then, the computing unit 222 determines the next possible motions that the person can make to the detected thing(s) and/or other person(s) on the basis of the correspondence information stored in the memory 221 (Step S203). The acceleration sensor 131 and the angular velocity sensor 132 measure acceleration and angular velocity, respectively (Step S204).

The computing unit 244 compares temporal changes in the acceleration and angular velocity measured by the acceleration sensor 131 and the angular velocity sensor 132 with respective output waveform patterns of acceleration and angular velocity corresponding to the possible motions determined by the computing unit 222 with reference to the memory 142 (Step S205). When the computing unit 244 has detected a part similar to any of the patterns (YES at Step S206), the computing unit 244 identifies a motion corresponding to the similar pattern as a motion that the person made (Step S207). On the other hand, if the computing unit 244 has not detected any part similar to any of the patterns (NO at Step S206), the process at Step S201 is again performed.

The transmitter 151 transmits a motion name of the motion identified by the computing unit 244 and current time obtained from the clock 143 to an external device (Step S208). Incidentally, such a motion identifying process is repeatedly performed.

Effect of Second Embodiment

The information processing apparatus 200 determines the next possible motions that a person can make to thing(s) and/or other person(s) located around person's present location, and compares temporal changes in measured measurement information with respective patterns of measurement information corresponding to the next possible motions, and, when having detected a part similar to any of the patterns, identifies a motion corresponding to the pattern as a motion that the person made. The information processing apparatus 200 targets only patterns of measurement information corresponding to the next possible motions for comparison with temporal changes in measured measurement information, and consequently can suppress a decrease in processing performance. If there are ten motions to be identified, and it takes 1 microsecond to identify each motion, it takes 10 microseconds to compare all patterns with temporal changes in measurement information; however, if the next possible motions are three motions, it takes only 3 microseconds.

### Third Embodiment

The embodiments of the information processing apparatus according to the present invention are explained above; however, besides the above-described embodiments, the present invention can be embodied in various different forms. Different embodiments of (1) the application of the information processing apparatus, (2) a configuration, and (3) a program are explained below.

(1) Application of Information Processing Apparatus

In the above embodiments, there is described the case where the information processing apparatus 100 or 200 is fitted on the abdomen of a person. However, the application of the information processing apparatuses 100 and 200 is not limited to the above-described application example. Specifically, the motion identifying process can be performed by acquiring information that identifies a person's motion from outside. For example, the measuring unit 130 can be set up outside the information processing apparatus, and the information processing apparatus can be realized as information equipment that receives measurement information from the external measuring unit 130 and performs the motion identifying process. Furthermore, record information and correspondence information of a motion and pattern information on output waveform patterns of acceleration and angular velocity, etc. can be stored in an external storage device, and the information processing apparatus can arbitrarily acquire information from the external storage device.

(2) Configuration

The processing procedures, control procedures, specific names, and information including various data and parameters illustrated in the above description and the drawings can be arbitrarily changed unless otherwise specified. Furthermore, components of each apparatus illustrated in the drawings are functionally conceptual ones, and do not always have to be physically configured as illustrated in the drawings. That is, the specific forms of division and integration of components of each apparatus are not limited to those illustrated in the drawings, and all or some of the components can be functionally or physically divided or integrated in arbitrary units depending on respective loads and use conditions, etc.

For example, the information processing apparatuses 100 and 200 can be integrated into one apparatus. The information processing apparatus 100 is useful in identifying a motion of a person who mostly works at the same place. The information processing apparatus 200 is useful in identifying a motion of a person who frequently moves over a wide range. Therefore, if the information processing apparatuses 100 and 200 are integrated into one apparatus, the above-described effects can be complemented.

Furthermore, the correspondence information is not limited to those illustrated in the drawings. Moreover, types and motion names of motions to be identified are not limited to those illustrated in the drawings. Furthermore, the incidence rate is not limited to either a “probable motion” or a “less-probable motion”; alternatively, the incidence rate can be divided into more categories, and the process can be performed according to the incidence rate.

(3) Program

As one mode, a motion identifying program executed by the information processing apparatus 100 or 200 is recorded on a computer-readable recording medium, such as a CD-ROM, a flexible disk (FD), a CD-R, or a digital versatile disk (DVD), in an installable or executable file format, and the recording medium is provided. Furthermore, the motion identifying program executed by the information processing apparatus 100 or 200 can be stored on a computer connected to a network such as the Internet, and the motion identifying program can be provided by causing a user to download it via the network. Moreover, the motion identifying program executed by the information processing apparatus 100 or 200 can be provided or distributed via a network such as the Internet. Furthermore, the motion identifying program can be built into a ROM or the like in advance.

The motion identifying program executed by the information processing apparatus 100 or 200 is composed of modules including the above-described units (the determining unit 120 or 220 and the identifying unit 140 or 240). A CPU (a processor) as actual hardware reads out the motion identifying program from a storage medium, and executes the motion identifying program, thereby the above units are loaded into the main memory, and the determining unit 120 or 220 and the identifying unit 140 or 240 are generated on the main memory.

According to one aspect of the present invention, it is possible to suppress a decrease in processing performance.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An information processing apparatus comprising:
a determining unit configured to determine possible motions that a person can make; and
an identifying unit configured to perform a pattern detecting process for detecting a pattern similar to measurement information measured according to a person's motion in patterns corresponding to the possible motions determined by the determining unit out of predetermined patterns of measurement information for person's motions, and identify a motion corresponding to the detected pattern as a motion that the person made.

2. The information processing apparatus according to claim 1, wherein
the determining unit determines the possible motions from an already-identified motion that the person made on the basis of correspondence information indicating correspondence of a person's motion record to possible motions.

3. The information processing apparatus according to claim 1, wherein
the determining unit detects thing(s) and/or other person(s) located in a predetermined range of area including person's present location in map information, and determines the possible motions from the detected thing(s) and/or other person(s) on the basis of correspondence information indicating correspondence of a thing or another person to possible motions.

4. The information processing apparatus according to claim 1, wherein
the determining unit determines respective incidence rates that represent the degrees of probability of the possible motions, and
the identifying unit performs the pattern detecting process so that the lower the incidence rate, the more simplified pattern detecting process the identifying unit performs.

5. The information processing apparatus according to claim 1, further comprising a measuring unit configured to measure measurement information, wherein
the identifying unit performs the pattern detecting process for detecting a pattern similar to the measurement information measured by the measuring unit.

6. The information processing apparatus according to claim 1, wherein
the measurement information is at least any one of acceleration and angular velocity, and
the identifying unit compares at least any one of temporal changes in acceleration and angular velocity measured according to a person's motion with predetermined patterns of acceleration and angular velocity for person's motions, and, when having detected a part similar to any of the patterns, identifies a motion corresponding to the pattern as a motion that the person made.

7. A motion identifying method comprising:
determining possible motions that a person can make; and
performing a pattern detecting process for detecting a pattern similar to measurement information measured according to a person's motion in patterns corresponding to the possible motions determined at the determining out of predetermined patterns of measurement information for person's motions, and identifying a motion corresponding to the detected pattern as a motion that the person made.

8. A non-transitory computer-readable recording medium that contains a motion identifying program causing a computer to execute:
determining possible motions that a person can make; and
performing a pattern detecting process for detecting a pattern similar to measurement information measured according to a person's motion in patterns corresponding to the possible motions determined at the determining out of predetermined patterns of measurement information for person's motions, and identifying a motion corresponding to the detected pattern as a motion that the person made.
